(19) United States
(12) Patent Application Publication
(10) Pub. No.: US 2025/0273088 A1
(43) Pub. Date: Aug. 28, 2025

(54) SIMULATOR BASED ON IMMERSIVE VIRTUAL REALITY FOR SURGICAL PROCEDURE TRAINING

(71) Applicant: UNIVERSIDAD DEL DESARROLLO, Santiago (CL)

(72) Inventors: José Ignacio GUZMÁN MONTOTO, Santiago (CL); Camilo Ignacio RODRÍGUEZ BELTRÁN, Santiago (CL); Santiago ESPINOSA PEÑA, Santiago (CL)

(21) Appl. No.: 18/868,156

(22) PCT Filed: May 24, 2023
(86) PCT No.: PCT/CL2023/050043

§ 371 Date: Nov. 21, 2024

(30) Foreign Application Priority Data
May 24, 2022 (CL) 1356-2022

(51) Int. Cl. G09B 19/00; G02B 27/01; G06F 3/01; G09B 5/06

(52) U.S. Cl. CPC G09B 19/00; G02B 27/0101; G02B 27/017; G06F 3/011; G09B 5/06; G02B 2027/0138; G02B 2027/0141

(57) ABSTRACT

The invention provides a system for carrying out virtual reality simulations of multiple surgeries for the training of health professionals, which is characterized in that it comprises: a mechanical interface in a case containing two control handles that simulate surgical clamps, and an electronic system that operatively connects to a screen for generating a visual interface containing developed graphics with motor skills exercises; and cameras and sensors that provide real-time information about the position and direction of the user's movements. According to the invention, the cameras are arranged in a virtual reality headset system and the sensors are arranged in the control handles, the cameras and sensors are operatively connected to software that allows a stereoscopic representation to be generated from images taken by the camera, and the visual interface comprises 3D objects having geometries similar to biological or surgical objects.

### TECHNICAL FIELD OF THE INVENTION

The present invention relates to the field of medicine or veterinary sciences and hygiene, specifically to the field of computer-assisted surgeries, or manipulators or robots specially adapted for use in surgery or simulators for the training of medical professionals. In particular, it provides an immersive and portable virtual reality simulator for training in minimal access surgeries and for the preparation of students or professionals in open surgery surgical techniques.

### BACKGROUND OF THE INVENTION

Within the field of health education, an important part is the training of health professionals and students of health-related careers by means of virtual reality simulations. These simulations are useful to teach trainees how to interact in a real surgery, improving their motor skills, spaces, and times associated with real surgery, without the need for a patient to be exposed to a learning error. In addition, students who participate in this type of simulations are able to face different scenarios, which helps to improve their ability to assess patients clinically and to make clinical decisions, as indicated in the scientific article by researchers from Universidad del Desarrollo (Telesimulation and teledebriefing to promote clinical reasoning in undergraduate medicine students; Marco Ortiz-Arévalo, Trinidad Campussano-Schialer, Álvaro Tolosa-Villarreal, Adriel Marco, Soledad Armijo-Rivera, Joaquín Díaz-Schmidt; Educación Médica; Volume 22, Issue 5, September-October 2021, Pages 283-286).

Currently, there are several devices that help surgeons to train for surgical interventions, helping to control certain maneuvers that must be performed in a real surgery and thus promoting the improvement of the surgeons' motor skills in these events. These devices have different mechanical and electronic configurations that help in a certain way to visualize the objects and components that interfere with or are used in or during an operation, but many of these current devices are focused on a particular type of surgery, for example, the existence of simulators for teaching the contents of childbirth and newborn in nursing, as described in the publication of Giselle Riquelme in her work entitled “Incorporation of simulation in teaching the contents of childbirth and newborn care in nursing” (Educación Médica Superior; Volume: 31, Issue: 4, Mar. 10, 2017). But many times, these simulations contain systems that are difficult to mobilize, leaving aside the practicality of devices that act as simulators and multidisciplinarity for different types of surgeries in a single piece of equipment.

An example of this type of system that simulates surgical operations under virtual reality is described in document CN106448403A which refers to a surgical training system for simulating thoracoscopic surgeries, which allows the surgeon to simulate various types of surgeries. The technical solution adopted and described in this document is a training system for simulating thoracoscopic surgery that includes a body, a monitor, a simulation endoscope, a simulation surgical instrument, and a control system; the monitor is fixed to the body through an arm, and the simulation endoscope and the simulation surgical instruments are connected to the body; the simulation endoscope and the simulation surgical instruments are provided with a rotation sensor and a movement sensor, and the control system is electrically connected. The control system includes a data receiving processing device, a 3D model processing server, and a 3D platform system server.

On the other hand, document ES2346025A1 refers to a simulation system for surgical training, which has technical particularities designed to allow training the trainee surgeon in the most realistic situation possible, without involving any simulation of material deterioration, and therefore allows unlimited repeatability in the same procedure. The trainee surgeon can observe not only the simulated situation as a real operation, but the system also allows to study any other aspect of this situation through different visualization modes. In addition, it allows customization of the practice by using patient data from diagnostic tests such as scanning, mapping and 3D CT, so that a trainee can check the status of an operation, decide where to place the portals of entry, and practice performing the surgery before performing the actual operation on said patient. This invention provides a solution to the learning the technique, which are critical and essential procedures in minimally invasive surgery.

However, the prior art is focused on improving aspects of specific surgeries, without the movement capacity of the system that generates the simulations, disregarding the possibility of training trainees in different places and of carrying out a possible remote evaluation of the trainee's performance, as well as not having different configurations for training under different circumstances that may arise in a real surgery.

Consequently, there is a need to create a portable system that helps surgeons and students to perfect the manipulation of surgical instruments and to acquire the necessary motor skills to avoid making mistakes in a real surgery, and where such a system is able to simulate various types of surgeries.

### SUMMARY OF THE INVENTION

The present invention provides an immersive virtual reality simulator-type system for minimal access surgery and other surgical procedures, characterized in that it comprises: a mechanical interface enclosed in a carrying case for portability that connects to a screen for simulation of surgical behavior; wherein said mechanical interface contains two handles that simulate surgical clamps for immersion of the user's senses; wherein said mechanical interface contains an electronic system that operatively connects to a screen to generate a visual interface; wherein said visual interface contains graphics developed with motor exercises; cameras and sensors that provide information on the position and orientation of the user's movements in real time, wherein said cameras are placed in a virtual reality headset system, such as Oculus Quest or HTC Vive, and sensors on the handles; wherein said cameras and sensors are operatively connected to a software that allows generating a stereoscopic representation from the images taken by the camera; and wherein said visual interface has three-dimensional objects with geometries similar to biological and surgical objects that may be involved in a surgery.

In a preferred embodiment of the invention, the virtual reality system is characterized in that said mechanical interface is included in a portable case.

In another preferred embodiment, the virtual reality system is characterized in that it contains a system for importing models of different possible surgeries.

In even a further preferred embodiment, the virtual reality system is characterized in that the headset contains complements to obtain a stereoscopic representation of the cameras.

In another preferred embodiment, the virtual reality system is characterized in that it contains a WEB architecture data storage system.

In a further preferred embodiment, the virtual reality system is characterized in that said software delivers physical parameters such as: position, orientation, distances, and velocities of the simulated clamps based on the maneuvers of the user on the handles.

In a more preferred embodiment, the surgical clamps system used in the present invention is characterized in that the rotation exerted by said clamps is based on a ball joint.

In yet another preferred embodiment, the virtual reality system is characterized in that it uses a three-dimensional gaming and simulation environment to recreate the training scenarios elaborated in simulation engines such as Unity 3D or Unreal.

In another preferred embodiment, the virtual reality system is characterized in that three-dimensional models are created in three-dimensional graphics editors such as 3dMax, Blender, Maya, among others, for said three-dimensional gaming and simulation environment.

In a different preferred embodiment, the virtual reality system is characterized in that it has a box containing three-dimensional models simulated in the three-dimensional gaming and simulation environment.

In a further preferred embodiment, the virtual reality system is characterized in that said container box has dimensions that will be in relation to the surgical procedure being simulated such as, for example, it has dimensions of 20 cm×20 cm×25 cm for simulations that contemplate gripping and carrying objects with surgical clamps.

In another preferred embodiment, the virtual reality system is characterized in that it includes a system for presenting results of the user's activity in the simulations, wherein said results are presented visually on the screen or sent via WEB.

The present invention also provides a device for obtaining virtual reality simulations of multiple surgeries, characterized in that it comprises: a mechanical interface that connects to a screen for visualizing the simulation of a surgical process; wherein said mechanical interface contains two handles that simulate surgical clamps for the immersion of the user's senses; and wherein said mechanical interface stores in an electronic device the information of the simulations performed.

### BRIEF DESCRIPTION OF THE FIGURES

FIG. 1 is a photograph of the simulator that is the object of the invention, when used by a surgical professional in a training center.

FIG. 2 illustrates the configuration of the parts that comprise the rotation of the clamp-type handle in an exploded view with a cross-sectional cut.

FIG. 3 illustrates the mechanical and geometrical configuration of the simulation mechanism of the surgical clamps in a profile view with a longitudinal cut.

FIG. 4 illustrates a schematic of the degrees of freedom that the clamps have in rotational movements, in a rectangular coordinate configuration.

FIG. 5 illustrates the internal components contained in the carrying case, showing a protective foam and the coupling of the simulator controllers.

FIG. 6 illustrates two photographs from different angles of the portable simulator, showing its components that include: the carrying case, the virtual reality headset, and the mechanical interface created to mimic the operation of the clamps attached to the headset controllers.

FIG. 7 illustrates a visualization of a simulation with some elements used during said simulation, such as: clamps and biological or surgical objects.

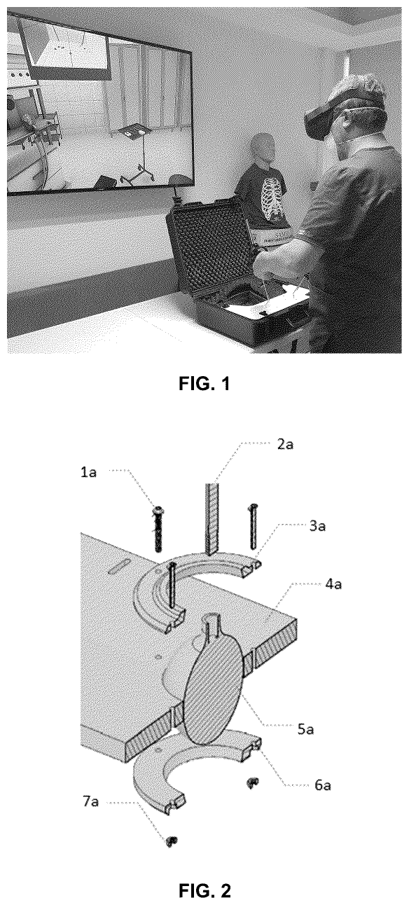

### DETAILED DESCRIPTION OF THE INVENTION

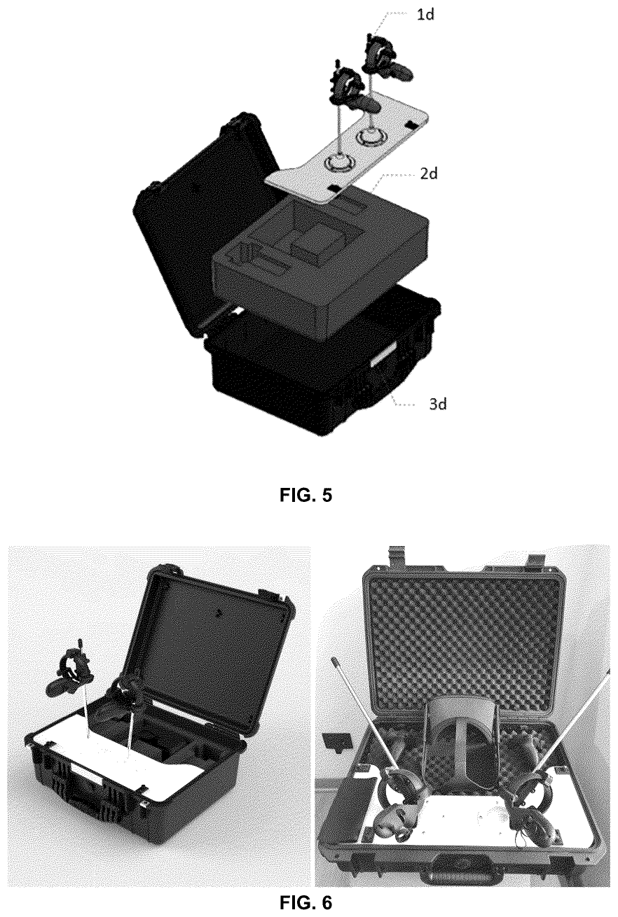

The present invention details a system and a device that offers a technological tool consisting of a system that performs virtual reality simulations for training in various surgeries, focused for use by students of medicine or other health-related careers, for practice and evaluation of their performance in such situations, as well as by health professionals for training before each new surgery, thus reducing preparation times, and improving motor skills and movements characteristic of different surgical interventions. This system and device offer the possibility of reducing the surgeon's adaptability times to each surgery, as well as, given its portability, enabling the training in different places, facilitating training from home and not necessarily from a hospital center. This technology is a case-type model (3d) as shown in FIG. 5, which contains an electronic system (1d) capable of being connected to a screen for its use, which is installed on a protective foam (2d).

The advantages of the system proposed in the present invention are its portability, since it allows its transportation from one place to another, in a case that includes the virtual reality device, and the electromechanical interface that is coupled to a worktable that is attached to it. The system does not include cables, as having a WIFI network for the training to be carried out will suffice, allowing mobility to the trainee, and if there is no connectivity, it can work in autonomous mode, which allows to send the training data in a deferred manner.

The system also allows the incorporation of courses for these surgical techniques. A face-to-face course is not necessary, since the instructor does not participate directly in the process. The system allows the trainee to go through stages with the simulator, which will provide feedback to the trainee and the course instructors. The use of the surgeon's real instruments is also not required, since they will be present virtually.

In particular, the immersive virtual reality simulator-type system for minimal access surgery of the invention is characterized in that it comprises: a mechanical interface that connects to a screen for simulation of surgical behavior; wherein said mechanical interface contains two handles that simulate surgical clamps for immersion of the user's senses; wherein said mechanical interface contains an electronic system that operatively connects to a screen to generate a visual interface; wherein said visual interface contains graphics developed with motor exercises; cameras and sensors that provide information on the position and orientation of the user's movements in real time, wherein said cameras are placed in a virtual reality headset system, such as Oculus Quest, and sensors on the handles; wherein said cameras and sensors are operatively connected to a software that allows generating a stereoscopic representation from the images taken by the camera; and wherein said visual interface has three-dimensional objects with geometries similar to biological and surgical objects that may be involved in a surgery.

In the context of the present invention, and by way of general clarification throughout the description, a handle will be understood as a system composed of several mechanical parts where the user of this invention interacts to simulate surgical clamps.

In a preferred embodiment, the virtual reality system is characterized in that said mechanical interface is included in a portable case.

In the context of the present invention, and by way of general clarification throughout the description, a carrying case will be understood as a case containing all the elements of the system of the present invention, to be moved and used wherever the user deems appropriate.

In the context of the present invention, and by way of general clarification throughout the description, a simulator assembly will be understood as the structure containing a base where the entire mechanical structure of the clamps and the electronics connecting to a screen is supported.

In the context of the present invention, and by way of general clarification throughout the description, a protective foam will be understood as a foam of variable geometry which is located inside the carrying case and which protects the entire structure of the simulator assembly.

In a preferred embodiment of the invention, the virtual reality system is characterized in that it contains a system for importing models of different possible surgeries.

In another preferred embodiment, the virtual reality system is characterized in that it contains a WEB architecture data storage system.

In a further preferred embodiment, the virtual reality system is characterized in that said software delivers physical parameters such as: position, orientation, distances, and velocities of the user.

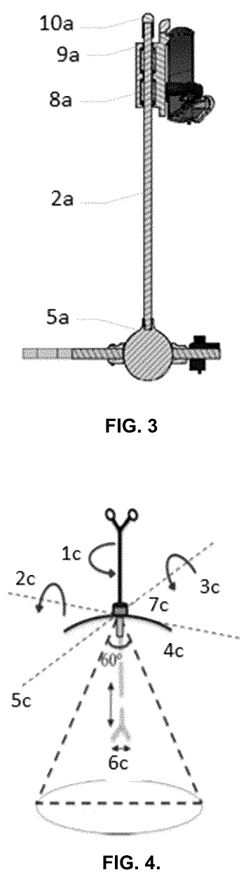

In a more preferred embodiment, the surgical clamp system used in the present invention is characterized in that the rotation exerted by said clamps is based on a ball joint (5a) as shown in FIG. 2, wherein a shaft (2a) is connected, coupled by rings (3a) (6a), on a support base plate (4a), which contains a fastening system with bolts (1a) and nuts (7a). FIG. 3 shows said coupling in the lower part of the entire structure in a profile view, while in the upper part it shows how the shaft (2a) is connected to the linear bearings (8a) where the user (controller) interacts, wherein said shaft in the upper part consists of a cap (10a) and a fastening system (9a).

In another preferred embodiment of the invention, the virtual reality system uses the Unity 3D gaming and simulation environment (Ronghai Wang, 6 abril 2017; A surgical training system for four medical punctures based on virtual reality and haptic feedback; 2017 IEEE Symposium on 3D User Interfaces (3DUI)) to make the most of the easiness of incorporating graphical features and physical behaviors.

In another preferred embodiment, the virtual reality system has the three-dimensional models of the objects to be used in the scene, all of these made in 3D Max (Li Yaqin, 22-24 Jun. 2010; The applying research for 3D mesh models watermarking based on 3D MAX; 2010 2nd International Conference on Education Technology and Computer). These are placed at the origin of the coordinate system and their vertices are updated according to the scale of the object from the Xform modifier (Mikko Honkala, July 2006; Multimodal interaction with xforms; Proceedings of the 6th International conference on Web engineering).

All the models to be loaded into the scene are checked for their import parameters, within these mainly the scale factor is configured, wherein a value of one is set, in order not to have scale changes and the use of animations.

In a different preferred embodiment, the virtual reality system is characterized in that it has a box containing three-dimensional models simulated in the three-dimensional gaming and simulation environment.

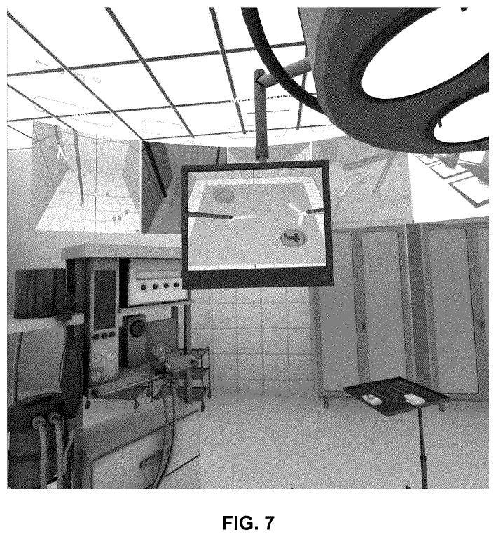

In a preferred embodiment, the box containing the virtual reality system of the invention may have variable dimensions depending on the surgical procedure to be simulated, and in a further preferred embodiment said container box has, for example, dimensions of 20 cm×20 cm×25 cm in the three-dimensional gaming and simulation environment as shown in FIG. 7.

In a preferred embodiment, the virtual reality system is characterized in that it contains a system for presenting results visually or sent via WEB.

The present invention also provides a device for obtaining virtual reality simulations of multiple surgeries, characterized in that it comprises: a mechanical interface that connects to a screen for visualization of the simulation of the behavior of a surgery; wherein said mechanical interface contains two handles that simulate surgical clamps for the immersion of the user's senses; wherein said mechanical interface is included in a portable case; and wherein said mechanical interface stores in an electronic device the information of the simulations.

The design of this product will allow solving the proposed problem through training in surgical medicine, which contributes to the creation of an adaptable tool in its use and transport, helping to improve the movements and motor skills of health professionals and students to improve the performance of surgical procedures.

### EXAMPLES OF EMBODIMENTS

### Example 1: Scene for Presenting the Simulation

The simulator has a persistent scene in charge of maintaining objects throughout the simulation, two exercise scenes, and one scene for presenting the results of the training sessions.

The persistent scene contains as a fundamental element the immersive camera system of the virtual reality headset, and attached to it, added in the same hierarchy of objects, are the right and left clamps. This guarantees that the virtual grippers can always be used when selecting another exercise scene. A description of each of these fundamental elements for the simulator (Unity gameobjects) is provided below:
OVRCameraRig: This is the tracking space of the virtual reality headset, which has a virtual reality camera that replaces the conventional camera of the Unity environment. It provides access to the interface with the hardware. It is observed how in this same element “child” is set, from the right controller to the prefabricated that contains the model of the right clamp.
ClampsLogic: It is the object that contains the necessary scripts for the operation of the clamps related to the manipulation of the objects of the scene. It includes codes developed in c # for: gripping and dropping elements; and swapping elements between the clamps.
GlobalSceneManager: It contains the controller codes (scripts) for loading and downloading scenes. It manages the control of exercise data storage in the server from APIs developed for this purpose.
NetworkManagerLogin: It is responsible for using the APIs developed to perform the user registration and authentication processes.

The coordination scene is created to perform a first practice exercise to allow the user to start getting familiar with the synchronized work between the hands and the direction of vision. It uses the environment of the container box and incorporates five spheres of a first color and five spheres of a second color. The spheres of the first color are to be “touched” by the left clamp and the spheres of the second color are to be touched by the right clamp. There is also a sphere of a third color, which if touched by either clamp, the exercise is concluded indicating that a serious error has occurred. The spheres behave inside the box according to a random movement in the horizontal plane. This scene also has a system for error detection that stores how many spheres have been touched with the wrong clamp or if a serious error has occurred. In addition, the time of the exercise is stored.

The scene of object gripping and transfer simulates one of the exercises most used by surgery students and tested in mechanical simulators. It consists of the inclusion in the box of a container that has five elements that must be taken and placed in another target container located at the end of the box. For these procedures, algorithms were developed that allow the gripping and transportation of physical elements, as well as the exchange of elements between the clamps. The simulator is capable of measuring the following parameters:
Distance traveled, for the right hand (meters).
Distance traveled, for the left hand (meters).
Economy of movement (optimal distance, data provided by teacher,/distance traveled), right hand.
Economy of movement (optimal distance, data provided by teacher,/distance traveled), left hand.
Average speed for the right hand (meters/sec).
Average speed for the left hand (meters/sec).
Position and orientation of the end of the clamps in each simulation frame.

The scene of presentation of the results aims to allow the participant of the simulation to observe a summary of their activity in the practices. This information is collected from the network and displayed on the device from a curved interface that allows observation of the data. Among these developed objects are:
ResultsManager: It controls the appearing of the tables and their filling from the information obtained from the server. It includes the ResultsManager.cs component (script), developed to include these functions.
CanvasTotalResults: It contains the tables to be displayed. These include the total data table, the coordination results table, and the results table of the object gripping and transfer exercise. This last object, which is expanded, has the results tables for coordination and gripping disabled, so when displayed in this scene only the total results will be displayed. During the simulation, when the user chooses to visualize the activity corresponding to these exercises, these tables are activated.

### Example 2: Design of the Mechanical Interface for the Simulation of the Rotational Behavior of the Clamps

The movement of the clamps in the simulation has to reach an imitation in such a way that this action leads to the immersion of the trainee's senses during training, bringing them closer to how they would perform this activity in a real surgical intervention. In this example, it was analyzed how many degrees of freedom are present in the process.

As can be seen in FIG. 4, there is a pivot point for the movement of the clamps which is at the entrance of the trocar (7c) in the abdominal cavity (4c). Three rotations in space are maintained during the actuation of the clamp, the first one around the longitudinal axis “Z” (Roll) (1c), which allows the rotation of the clamp, and the other two on the axes “X” (2c) and “Y” (3c) (Pitch and Yaw), which allows the inclination supported at the point of articulation. In addition, there are two other movements: one longitudinal, which characterizes the moving of the instrument, and the opening of the clamp, which allows the gripping of elements in the cavity. To solve this problem, a ball joint (5a) is created, which enables the Pitch and Yaw rotations described in FIG. 4. The ball joint can be observed, which is included in the support base and fastened by two rings, one upper and one lower, to this base. These rings are attached by means of a screw (1a) and a nut (7a) to the support base plate (4a).

The opening and closing are mimicked with the same device controller, performing actions from the software to capture the movement, so the design effort is directed to solve the longitudinal movement and rotation mentioned above.

### Example 3: Design of The Mechanical Interface for the Simulation of the Translational Behavior of the Clamps

The longitudinal movement, as seen in FIG. 4, is carried out by moving the shaft of the clamp over the trocar (7), forming a cylindrical joint. Many simulators mimic this process in a similar manner; however, this leads to a larger volume caused by the space that must be left for these clamps to move freely.

As portability is a design requirement, in order to maintain limited dimensions and avoid large parts such as the clamps, the movement of the hands and not of the clamps was used as a concept, that is, a reverse process. In the invention, a shaft or a rod is not introduced, but the simulated clamps are moved using the same shaft that supports them. In this way, a similar effect was obtained in the simulation, but achieving a reduction in the space used in the depth with respect to the original object.

It is observed in FIG. 3 how the shaft (2a) starts from the ball joint (5a) upwards and in this case the controller (2), which mimics the clamp support, moves through this element in a longitudinal movement that allows the surgeon to access any location in the virtual training volume. In the same FIG. 3 it is observed how linear bearings (8a) are the connection between the shaft and the user's hands. This connection guarantees the movement in depth and the rotation of the instrument around its axis (Roll).

The surgical clamps were modeled so that the depth of the simulation coincided with the largest dimension of the shaft, and the area corresponding to the object gripping section was built separately and integrated into the hierarchy as an additional component. The latter facilitates the subsequent rotation of the element with respect to the pivot point. Collision zones were also added to these clamps to allow the simulation of contact with other elements and the gripping and transportation of these in case the exercise requires it.

### Example 4: Materials and Construction System Inside the Simulator Assembly

The handles that allow movement and simulate the clamps used in surgery are made of 8 mm solid stainless steel bars, which ensures their resistance to mechanical tensile stresses or possible drops when installed on the ball joints. The connection of the bars to the ball joints is by means of a thread, thus ensuring a neat connection and always in the same position. As a last element, linear bearings were considered to generate a smooth and unobstructed sliding, thus simulating the normal working context.

The fastening system for the virtual reality headset controls was designed and constructed using three anchor points, thus ensuring correct positioning. These points were made with an anti-slip material, to provide a better grip in the turning and translation efforts that occur in the daily use of the simulator.

The grip was designed in such a way that it maintains the alignment of the coordinate system of the controller with the shaft of the handle used for movement and ensuring that there is no free play in any of the shafts where the user applies force.

1. An immersive virtual reality simulator-type system for minimal access surgery and other surgical procedures, CHARACTERIZED in that it comprises:
a mechanical interface that connects to a screen for simulating a surgical procedure;
said mechanical interface contains two handles that simulate surgical clamps for immersion of the user's senses;
said mechanical interface contains an electronic system that operatively connects to a screen to generate a visual interface;
wherein said visual interface contains graphics developed with motor exercises;
cameras and sensors that provide information on the position and orientation of the user's movements in real time, wherein said cameras are placed in a virtual reality headset system, and the sensors are placed in the handles;
wherein said cameras and sensors are operatively connected to a software for generating a stereoscopic representation from the images taken by the camera; and
wherein said visual interface has three-dimensional objects of geometries similar to biological and surgical objects.

2. The system of claim 1, CHARACTERIZED in that said mechanical interface is included in a portable case.

3. The system of claim 1, CHARACTERIZED in that it includes a system for importing models of different types of surgeries.

4. The system of claim 1, CHARACTERIZED in that the virtual reality headset contains internal electronic complements for stereoscopic representation of the cameras.

5. The system of claim 1, CHARACTERIZED in that it includes a WEB architecture data storage system.

6. The system of claim 1, CHARACTERIZED in that said software delivers information of physical parameters related to the position, orientation, distance, and velocities of the simulated clamps based on the maneuvers of the user on the handles.

7. The system of claim 1, CHARACTERIZED in that it uses a three-dimensional gaming and simulation environment created by means of simulation engines.

8. The system of claim 7, CHARACTERIZED in that it includes three-dimensional models on said three-dimensional gaming and simulation environment.

9. The system of claim 7, CHARACTERIZED in that it also includes a box containing three-dimensional models simulated in the three-dimensional gaming and simulation environment.

10. The system of claim 9, CHARACTERIZED in that said container box has dimensions in correspondence with the surgical procedure being simulated.

11. The system of claim 1, CHARACTERIZED in that it includes a system for presenting results of the user's mobility in the simulations, wherein said results are presented visually on the screen or sent via WEB.

12. A device for simulation of minimal access surgeries by means of immersive virtual reality, CHARACTERIZED in that it comprises: a mechanical interface that connects to a screen for visualizing the simulation of a surgery process; wherein said mechanical interface contains two handles that simulate surgical clamps for immersion of the user's senses; and wherein said mechanical interface stores in an electronic device the information of the simulations performed.

13. The device of claim 10, CHARACTERIZED in that said mechanical interface is included in a portable case.
